(19) United States
(12) Patent Application Publication
(10) Pub. No.: US 2015/0080329 A1
(43) Pub. Date: Mar. 19, 2015

(54) PHARMACEUTICAL COMPOSITIONS COMPRISING DHA, EPA, AND/OR GLA AND AN ANTIBIOTIC AGENT AND METHODS OF USE THEREOF

(71) Applicant: Dignity Sciences Limited, Leopardstown (IE)

(72) Inventors: Mehar Manku, Birmingham (GB); John Climax, Leopardstown (IE); Jonathan Rowe, Waterford, CT (US)

(21) Appl. No.: 14/491,000

(22) Filed: Sep. 19, 2014

Related U.S. Application Data
(60) Provisional application No. 61/879,891, filed on Sep. 19, 2013.

(51) Int. Cl. A61K 31/7036; A61K 31/202

(52) U.S. Cl. CPC A61K 31/7036; A61K 31/202
USPC 514/39; 514/560

(57) ABSTRACT

The present disclosure provides compositions comprising fatty acids, or derivatives thereof (e.g., C1-C4 esters) including, for example, DHA, EPA and/or GLA, used in combination with one or more antibiotic agents for the treatment of disease and/or disorders such as a skin or ocular infection.

### FIELD

The disclosure generally relates to compositions comprising fatty acids including, for example, docosahexaenoic acid (DHA), eicosapentaenoic acid (EPA) and/or gamma-linolenic acid (GLA), alone or in combination with one or more antibiotic agents for the treatment of disease and/or disorders such as a skin or ocular infection.

### BACKGROUND

Scores of different bacterial species colonize the skin and other organs as normal flora. However, when the skin's normal continuity or other tissue microenvironment becomes disrupted, the resulting contusions, wounds, lesions, incisions, pockets, lacerations, and/or other disruptions can become infected by a wide variety of bacterial species, some of which have become or are rapidly becoming resistant to existing therapies. Accordingly, there exists a need for compositions that are more effective in the treatment of skin and oral infections.

### SUMMARY

The present disclosure provides compositions comprising one or more long-chain polyunsaturated fatty acids agents (LC-PUFA) including, for example, DHA, EPA and/or GLA, used alone or in combination with antibiotic agents for the treatment of disease and/or disorders of the skin, eye or other organs.

The present disclosure also provides methods for treating or preventing skin and ocular infections in a subject in need thereof comprising administering to the subject a pharmaceutical composition comprising a therapeutically effective amount of DHA, EPA, or GLA or combinations thereof alone or in combination with one or more antibiotic agents. In some embodiments, the pharmaceutical composition comprises about 0.1 wt. % to about 20 wt. % of DHA, EPA, or GLA.

In some embodiments, the pharmaceutical composition comprises a sub-therapeutic amount of one or more of DHA, EPA, or GLA along with a therapeutic amount of one or more antibiotic agents. In some embodiments, the pharmaceutical composition comprises a therapeutic amount of one or more of DHA, EPA, or GLA along with a sub-therapeutic amount of one or more antibiotic agents. In some embodiments, the pharmaceutical composition comprises a sub-therapeutic amount of one or more of DHA, EPA, or GLA along with a sub-therapeutic amount of one or more antibiotic agents.

In some embodiments, the pharmaceutical composition comprises one or more pharmaceutically acceptable excipients.

In some embodiments, the one or more antibiotic agents are selected from the group consisting of: neomycin sulfate, polymyxin B, bacitracin zinc, β-lactams (e.g., ampicillin, amoxicillin, imipenem, meropenem), carbapenems, cephalosporins (e.g., cephalexin, cephalothin, cefalozin, cefuroxime, cefotaxime, ceftazidime), fluoroquinolones, oxazolidinones, lincosamides, metronidazole, macrolide antibiotics (e.g., clindamycin, erythromycin), quinolone antibiotics (e.g., levofloxacin, ciprofloxacin), penicillins, glycopeptides (e.g., vancomycin), aminoglycosides (e.g., neomycin, gentamicin, tobramycin), trimethoprim/sulfamethoxazole (also known as co-trimoxazole or TMP/SMX), doxycycline, triclosan, metronidazole, monocycline and tetracycline.

In some embodiments, the one or more antibiotic agents are selected from the group consisting of: neomycin salts (e.g., neomycin sulfate), polymixin B, and/or bacitracin salts (e.g., bacitracin zinc). In some embodiments, the one or more antibiotic agents are neomycin sulfate, polymyxin B and bacitracin zinc.

In some embodiments, the step of administering comprises topically applying the composition to an area of the skin or eye afflicted with lesions. As used herein, the term “lesion” refers broadly to any disruption in the normal continuity and function of the skin, eye or other tissue and includes, for example, contusions, wounds, burns, sores, ulcers, scrapes, incisions, lacerations, conjunctivitis, stye, blepharitis, skin infection, gingivitis and periodontal disease. In some embodiments, the area of the skin afflicted with lesions is washed prior to application of the pharmaceutical composition. In some embodiments, the lesions are inflammatory type and/or non-inflammatory type lesions.

In some embodiments, applying the composition results in about a 10%, 20%, 30%, 40%, 50%, 60%, 70%, 80%, 90% or more reduction in the lesion.

In some embodiments, the lesion is associated with gram positive bacteria, gram negative bacteria or fungi.

In some embodiments, the lesion is associated with one or more of: Staphylococcus spp., Propionibacterium spp., Staphylococcus spp., Streptococcus spp, Corynebacterium spp., Porphyromonas spp. Micrococcus spp., Pseudomonas aeruginosa, Pasteurella multocida, Capnocytophaga canimorsus, Bartonella spp., Klebsiella rhinoscleromatis, Helicobacter spp, Aspergillus niger, Aureobasiduim pullulans, Chaetomium globosum, Gliocladium virens, Penicillum funiculosum, Candida albicans, Saccharomyces cerevisiae and Vibrio vulnificus

In some embodiments, the pharmaceutical composition is administered to the subject once a day, twice a day, or three times a day.

In some embodiments, the pharmaceutical composition is a cream, lotion, gel or emulsion.

In some embodiments, the subject previously exhibited lesions.

The present disclosure also provides methods of treating or preventing a skin or ocular infection in a subject in need thereof comprising administering to the subject a pharmaceutical composition comprising a therapeutically effective amount of DHA. In some embodiments, the pharmaceutical composition comprises about 0.1% to about 20 wt. % of DHA.

In some embodiments, the step of administering comprises topically applying the composition to an area of the skin, eye or gingiva afflicted with lesions. In some embodiments, the area of the skin afflicted with lesions is first washed prior to application of the pharmaceutical composition.

In some embodiments, the composition reduces about 10%, 20%, 30%, 40%, 50%, 60%, 70%, 80%, 90% or more of the lesion.

In some embodiments, the lesion is associated with one or more of: Staphylococcus spp., Propionibacterium spp., Streptococcus spp, Corynebacterium spp., Porphyromonas spp. Micrococcus spp., Pseudomonas aeruginosa, Pasteurella multocida, Capnocytophaga canimorsus, Bartonella spp., Klebsiella rhinoscleromatis, Helicobacter spp., Aspergillus niger, Aureobasiduim pullulans, Chaetomium globosum, Gliocladium virens, Penicillum funiculosum, Candida albicans, Saccharomyces cerevisiae and Vibrio vulnificus

In some embodiments, the pharmaceutical composition is administered to the subject once a day, twice a day, or three times a day.

In some embodiments, the pharmaceutical composition is a cream, lotion, gel, rinse, paste or emulsion.

In some embodiments, the subject previously exhibited a lesion.

The present disclosure also provides compositions for use in treating a skin or oral infection comprising a therapeutically effective amount of DHA, EPA, or GLA. In some embodiments, the composition comprises about 0.1% to about 20 wt. % of DHA, EPA, or GLA.

The present disclosure also provides methods for treating or preventing bacterial infection on the skin, eye or other tissue comprising applying to the lesion one or more of DHA, EPA, or GLA. In some embodiments, the composition comprises about 0.1% to about 20 wt. % of DHA, EPA, or GLA.

The present disclosure also provides methods for improving the antimicrobial activity of an agent used in the treatment or prevention of skin or ocular infections comprising adding a composition comprising one or more of DHA, EPA, or GLA to the agent. In some embodiments, the agent used in the treatment or prevention of skin or ocular infections is an antibiotic agent. In some embodiments, the composition comprises about 0.1% to about 20 wt. % of DHA, EPA, or GLA.

The present disclosure also provides methods of inhibiting one or more skin or ocular pathogens including, for example, its reproduction, growth or recolonization, comprising contacting the one or more skin or oral pathogens with a composition comprising DHA, EPA, or GLA. In some embodiments, the composition comprises about 0.1% to about 20 wt. % of DHA, EPA, or GLA.

In some embodiments, the methods may further comprise administering to the subject a steroid. In some embodiments, the steroid is a corticosteroid such as hydrocortisone, prednicarbate, fluticasone and derivatives thereof, or mometasone and derivatives thereof.

In some embodiments, the subject is administered the therapeutically effective amount of DHA, EPA, or GLA, the one or more antibiotic agents, and the steroid concomitantly.

In some embodiments, the pharmaceutical composition comprises about 0.1 wt. % to about 20 wt. % of DHA, EPA, or GLA.

In some embodiments, the pharmaceutical compositions described herein comprise one or more of steareth-2, steareth-21, cetyl alcohol, ascorbyl palmitate, about a-tocopherol, medium-chain triglycerides (e.g., Crodamol GTCC), myristyl myristate, isopropryl palmitate, glycerin, phenoxyethanol, ascorbic acid, carbomer, xanthan gum, liquid soy lecithin, and/or Mild Care 345 fragrance.

In some embodiments, the step of administering comprises topically applying the composition to an area afflicted with contusions, wounds, burns, sores, ulcers, scrapes, incisions, lacerations, skin infection, eye infection, gingivitis or periodontal disease.

In some embodiments, the pharmaceutical composition is administered to the subject once a day, twice a day, or three times a day.

In some embodiments, the pharmaceutical composition is a cream, lotion, gel, sterile liquid solution or paste.

The present disclosure also provides methods for improving the efficacy of an agent used in the treatment of contusions, wounds, burns, sores, ulcers, scrapes, incisions, lacerations, skin infection, ocular or periodontal disease comprising adding a therapeutically effective amount of DHA, EPA, or GLA to the agent. In some embodiments, the agent is one or more antibiotic agents.

In some embodiments, about 0.1% to about 20 wt. % of DHA, EPA, or GLA is added to the agent.

The present disclosure also provides methods for reducing the efficacious dose of an agent used in the treatment of contusions, wounds, burns, sores, ulcers, scrapes, incisions, lacerations, conjunctivitis, stye, blepharitis, skin infection, gingivitis or periodontal disease comprising adding a therapeutically effective amount of DHA, EPA, or GLA to the agent.

In some embodiments, about 0.1% to about 20 wt. % of DHA, EPA, or GLA is added to the agent.

These and other embodiments of the invention are described in further detail below.

### DETAILED DESCRIPTION

The present disclosure provides compositions (e.g., pharmaceutical compositions) and formulations that comprise fatty acid agents including, for example, DHA, EPA and/or GLA alone; or with one or more antibiotic agents, for example, neomycin, polymyxin B, and/or bacitracin zinc. Such agents have been found to reduce including, inhibit, the growth of bacteria associated with skin and ocular infections such as Staphylococcus spp. (including, for example, S. aureus, S. lugdunensis, S. schleiferi and other coagulase-negative Staphylococcus spp.), Streptococcus spp. (including, for example, β-haemolytic Streptococci, Viridans group Streptococci, non-haemolytic Streptococci, and Streptococcus milleri group), Corynebacterium spp., Bacillus spp. (including, for example, B. anthracis and B. cereus), Acinetobacter spp., Moraxella spp., Peptostreptococcus spp., Propionibacterium spp., (including, for example, P. Acnes), Candida spp., Pseudomonas spp. and other non-fermentative bacilli (including, for example, P. aeruginosa), Dermatophytes, Enterobacteriaceae, Pasturella multocida, Mycobacterium spp., Haemophilus spp., Nocardia spp., Erysipelothrix rhusiopathiae, Vibrio spp., Enterococcus spp., Eikenella corrodens, anaerobes, Corynebacterium spp., Actinomyces spp., and fungal pathogens. Furthermore, the inventors have found that, in many cases, the use of these fatty acid agents in combination with existing antibacterial agents (e.g., nicotinamide, benzoyl peroxide, adapalene, metronidazole, neomycin, polymyxin B, bacitracin zinc, β-lactams (e.g., ampicillin, amoxicillin, imipenem, meropenem), carbapenems, cephalosporins (e.g., cephalexin, cephalothin, cefalozin, cefuroxime, cefotaxime, ceftazidime), fluoroquinolones, oxazolidinones, lincosamides, macrolide antibiotics (e.g., clindamycin, erythromycin), quinolone antibiotics (e.g., levofloxacin, ciprofloxacin), penicillins, triclosan, monocycline, glycopeptides (e.g., vancomycin), aminoglycosides (e.g., neomycin, gentamicin, tobramycin), trimethoprim/sulfamethoxazole (also known as co-trimoxazole or TMP/SMX), doxycycline and tetracycline) provides additional reduction in the growth of bacteria compared to each agent used singly. Given their capacity to reduce, inhibit and/or prevent, the growth of bacteria, the compositions and formulations disclosed herein may be used in the treatment of disease and/or disorders associated with the growth of bacteria.

The present disclosure provides compositions comprising fatty acids including, for example, DHA, EPA and/or GLA in free acid or derivative form, used in combination with antibacterial agents including, for example, nicotinamide, benzoyl peroxide, adapalene, metronidazole, neomycin, polymyxin B, and bacitracin zinc and triclosan. In some embodiments, the compositions comprise about 0.1 wt. % to about 20 wt. % of DHA, EPA, or GLA or derivative thereof. Contemplated combinations include, without limitation, DHA and neomycin sulfate; EPA and neomycin sulfate; GLA and neomycin sulfate; DHA and polymyxin B; EPA and polymyxin B; GLA and polymyxin B; DHA and bacitracin zinc; EPA and bacitracin zinc; and GLA and bacitracin zinc; DHA, neomycin sulfate, polymyxin B and bacitracin zinc; EPA neomycin sulfate, polymyxin B and bacitracin zinc; and GLA neomycin sulfate, polymyxin B and bacitracin zinc. In some embodiments, a composition comprising DHA, EPA and/or GLA includes a therapeutically effective amount of neomycin sulfate. In some embodiments, a composition comprising DHA, EPA and/or GLA includes a therapeutically effective amount of polymyxin B. In some embodiments, a composition comprising DHA, EPA and/or GLA includes a therapeutically effective amount of bacitracin zinc.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

Docosahexaenoic acid, also referred to as (4Z,7Z,10Z,13Z,16Z,19Z)-docosa-4,7,10,13,16,19-hexaenoic acid or C 22:6ω3 (“DHA”), is a naturally occurring omega-3 fatty acid mostly derived from fish oil. As used herein, the term “DHA” refers to DHA free acid and/or a pharmaceutically acceptable ester, derivative, conjugate or salt thereof, or mixtures of any of the foregoing. In some embodiments, DHA is in the form of a C₁₋₄ alkyl ester such as methyl ester or ethyl ester form.

Eicosapentaenoic acid is also referred to as all-cis-5,8,11,14,17-eicosapentaenoic acid or C 20:5ω3 (“EPA”). EPA is a naturally occurring omega-3 fatty acid mostly derived from fish oil. As used herein, the term “EPA” refers to EPA in its free acid form and/or a pharmaceutically acceptable ester, derivative, conjugate or salt thereof, or mixtures of any of the foregoing. In some embodiments, the EPA is in the form of a C₁₋₄ alkyl ester such as methyl ester or ethyl ester form.

Gamma-linolenic acid, also referred to as all-cis-6,9,12-octadecatrienoic acid, or C 18:3ω6 (“GLA”), is a component of natural oils from a variety of plants such as Echium, blackcurrant, borage, evening primrose, hackelia, trichodesma, and buglossoides, to name a few. As used herein, the term “GLA” refers to GLA in its free acid form and/or a pharmaceutically acceptable ester, derivative, conjugate or salt thereof, or mixtures of any of the foregoing.

In various embodiments, the invention provides pharmaceutical compositions, for example topically deliverable compositions, comprising one or more of DHA, EPA, GLA or mixtures thereof.

In one embodiment, the present disclosure provides pharmaceutical compositions comprising, for example, an amount (e.g., a therapeutically effective amount) of DHA, EPA, GLA, or a combination thereof. In one embodiment, the pharmaceutical composition comprises about 0.1 wt. % to about 20 wt. % of the DHA, EPA, GLA, or a combination thereof, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, about 10 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13 wt. %, about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, about 14 wt. %, about 14.1 wt. %, about 14.2 wt. %, about 14.3 wt. %, about 14.4 wt. %, about 14.5 wt. %, about 14.6 wt. %, about 14.7 wt. %, about 14.8 wt. %, about 14.9 wt. %, about 15 wt. %, about 15.1 wt. %, about 15.2 wt. %, about 15.3 wt. %, about 15.4 wt. %, about 15.5 wt. %, about 15.6 wt. %, about 15.7 wt. %, about 15.8 wt. %, about 15.9 wt. %, about 16 wt. %, about 16.1 wt. %, about 16.2 wt. %, about 16.3 wt. %, about 16.4 wt. %, about 16.5 wt. %, about 16.6 wt. %, about 16.7 wt. %, about 16.8 wt. %, about 16.9 wt. %, about 17 wt. %, about 17.1 wt. %, about 17.2 wt. %, about 17.3 wt. %, about 17.4 wt. %, about 17.5 wt. %, about 17.6 wt. %, about 17.7 wt. %, about 17.8 wt. %, about 17.9 wt. %, about 18 wt. %, about 18.1 wt. %, about 18.2 wt. %, about 18.3 wt. %, about 18.4 wt. %, about 18.5 wt. %, about 18.6 wt. %, about 18.7 wt. %, about 18.8 wt. %, about 18.9 wt. %, about 19 wt. %, about 19.1 wt. %, about 19.2 wt. %, about 19.3 wt. %, about 19.4 wt. %, about 19.5 wt. %, about 19.6 wt. %, about 19.7 wt. %, about 19.8 wt. %, about 19.9 wt. %, or about 20 wt % of the DHA, EPA, GLA, or a combination thereof.

In one embodiment, the pharmaceutical composition further comprises an additional active agent. In one embodiment, the pharmaceutical composition comprises an amount of the additional active agent that is less than the generally recognized therapeutically effective amount for that agent. In one embodiment, the pharmaceutical composition comprises an amount of the additional active agent that is equal to or greater than the generally recognized therapeutically effective amount for that agent. In one embodiment, the additional active agent has not previously been recognized as effective in the treatment or prevention of skin or ocular infections. In another embodiment, the additional active agent is approved for use in the treatment or prevention of skin or ocular infections. In one embodiment, the additional active agent is an antibiotic agent.

In one embodiment, the additional active agent is neomycin or its derivative neomycin sulfate (also referred to as (2R,3S,4R,5R,6R)-5-amino-2-(aminomethyl)-6-[(1R,2R,3S,4R,6S)-4,6-diamino-2-[(2S,3R,4S,5R)-4-[(3R,4R,5S,6S)-3-amino-6-(aminomethyl)-4,5-dihydroxyoxan-2-yl]oxy-3-hydroxy-5-(hydroxymethyl)oxolan-2-yl]oxy-3-hydroxycyclohexyl]oxyoxane-3,4-diol, sulfuric acid). In one embodiment, the pharmaceutical composition comprises an amount of neomycin sulfate that is less than the generally recognized therapeutically effective amount. In one embodiment, the pharmaceutical composition comprises an amount of neomycin sulfate that is equal to or greater than the generally recognized therapeutically effective amount. In one embodiment, the pharmaceutical composition comprises about 0.5 mg to about 10 mg of neomycin sulfate per gram of pharmaceutical composition, for example about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, or about 10 mg of neomycin sulfate per gram of pharmaceutical composition. In one embodiment, the pharmaceutical composition comprises less than about 3.5 mg of neomycin sulfate per gram of pharmaceutical composition. In one embodiment, the pharmaceutical composition comprises about 3.5 mg of neomycin sulfate per gram of pharmaceutical composition. In one embodiment, the pharmaceutical composition comprises more than about 3.5 mg of neomycin sulfate per gram of pharmaceutical composition. In one embodiment, the pharmaceutical composition comprises no neomycin sulfate.

In one embodiment, the additional active agent is polymyxin B, a mixture of polymyxin B1 and polymyxin B2 (also referred to as Aerosporin, PMB, and N-[4-amino-1-[[1-[[4-amino-1-oxo-1-[[6,9,18-tris(2-aminoethyl)-15-benzyl-3-(1-hydroxyethyl)-12-(2-methylpropyl)-2,5,8,11,14,17,20-heptaoxo-1,4,7,10,13,16,19-heptazacyclotricos-21-yl]amino]butan-2-yl]amino]-3-hydroxy-1-oxobutan-2-yl]amino]-1-oxobutan-2-yl]-6-methyloctanamide). In one embodiment, the pharmaceutical composition comprises an amount of polymyxin B that is less than the generally recognized therapeutically effective amount. In one embodiment, the pharmaceutical composition comprises an amount of the polymyxin B that is equal to or greater than the generally recognized therapeutically effective amount. In one embodiment, the pharmaceutical composition comprises about 1,000 units to about 20,000 units of polymyxin B per gram of pharmaceutical composition (about 119 μg to about 2.38 mg of polymyxin B per gram of pharmaceutical composition), for example about 1,000 units, about 1,500 units, about 2,000 units, about 2,500 units, about 3,000 units, about 3,500 units, about 4,000 units, about 4,500 units, about 5,000 units, about 5,500 units, about 6,000 units, about 6,500 units, about 7,000 units, about 7,500 units, about 8,000 units, about 8,500 units, about 9,000 units, about 9,500 units, about 10,000 units, about 10,500 units, about 11,000 units, about 11,500 units, about 12,000 units, about 12,500 units, about 13,000 units, about 13,500 units, about 14,000 units, about 14,500 units, about 15,000 units, about 15,500 units, about 16,000 units, about 16,500 units, about 17,000 units, about 17,500 units, about 18,000 units, about 18,500 units, about 19,000 units, about 19,500 units, or about 20,000 units of polymyxin B per gram of pharmaceutical composition. In one embodiment, the pharmaceutical composition comprises less than about 5,000 units of polymyxin B per gram of pharmaceutical composition. In one embodiment, the pharmaceutical composition comprises less than about 10,000 units of polymyxin B per gram of pharmaceutical composition. In one embodiment, the pharmaceutical composition comprises more than about 5,000 units of polymyxin B per gram of pharmaceutical composition. In one embodiment, the pharmaceutical composition comprises more than about 10,000 units of polymyxin B per gram of pharmaceutical composition. In one embodiment, the pharmaceutical composition comprises about 5,000 units of polymyxin B per gram of pharmaceutical composition. In one embodiment, the pharmaceutical composition comprises about 6,500 units of polymyxin B per gram of pharmaceutical composition. In one embodiment, the pharmaceutical composition comprises about 10,000 units of polymyxin B per gram of pharmaceutical composition. In one embodiment, the pharmaceutical composition comprises no polymyxin B.

In one embodiment, the additional active agent is bacitracin zinc. In one embodiment, the pharmaceutical composition comprises an amount of bacitracin zinc that is less than the generally recognized therapeutically effective amount. In one embodiment, the pharmaceutical composition comprises an amount of the bacitracin zinc that is equal to or greater than the generally recognized therapeutically effective amount. In one embodiment, the pharmaceutical composition comprises about 100 units to about 800 units of bacitracin zinc (about 2.5 mg to about 20 mg of bacitracin zinc per gram of pharmaceutical composition), for example about 100 units, about 125 units, about 150 units, about 175 units, about 200 units, about 225 units, about 250 units, about 275 units, about 300 units, about 325 units, about 350 units, about 375 units, about 400 units, about 425 units, about 450 units, about 475 units, about 500 units, about 525 units, about 550 units, about 575 units, about 600 units, about 625 units, about 650 units, about 675 units, about 700 units, about 725 units, about 750 units, about 775 units, or about 800 units of bacitracin zinc per gram of pharmaceutical composition. In one embodiment, the pharmaceutical composition comprises less than about 400 units of bacitracin zinc per gram of pharmaceutical composition. In one embodiment, the pharmaceutical composition comprises about 400 units of bacitracin zinc per gram of pharmaceutical composition. In one embodiment, the pharmaceutical composition comprises more than about 400 units of bacitracin zinc per gram of pharmaceutical composition. In one embodiment, the pharmaceutical composition comprises less than about 500 units of bacitracin zinc per gram of pharmaceutical composition. In one embodiment, the pharmaceutical composition comprises about 500 units of bacitracin zinc per gram of pharmaceutical composition. In one embodiment, the pharmaceutical composition comprises more than about 500 units of bacitracin zinc per gram of pharmaceutical composition. In one embodiment, the pharmaceutical composition comprises no bacitracin zinc.

In one embodiment, the additional active agents are neomycin sulfate, polymyxin B and bacitracin zinc. In one embodiment, the pharmaceutical composition comprises an amount of one or more of neomycin sulfate, an amount of polymyxin B and/or an amount of bacitracin zinc that is less than the generally recognized therapeutically effective amount. In one embodiment, the pharmaceutical composition comprises an amount of neomycin sulfate, an amount of polymyxin B and an amount of bacitracin zinc that are each less than the generally recognized therapeutically effective amount. In one embodiment, one gram of pharmaceutical composition comprises about 3.5 mg of neomycin sulfate, about 5,000 units of polymyxin B, and about 400 units of bacitracin zinc. In one embodiment, one gram of pharmaceutical composition comprises about 3.5 mg of neomycin sulfate, about 10,000 units of polymyxin B, and about 500 units of bacitracin zinc. In one embodiment, one gram of pharmaceutical composition comprises about 3.5 mg of neomycin sulfate, about 10,000 units of polymyxin B, and no bacitracin zinc.

In one embodiment, the additional active ingredient is triclosan. In one embodiment the pharmaceutical composition comprises 0.1-1% triclosan. In another embodiment the pharmaceutical composition comprises an amount of triclosan that is less than the generally recognized therapeutically effective amount.

In one embodiment, the pharmaceutical composition further comprises an analgesic agent. In one embodiment, the analgesic agent is a topical or systemic analgesic. In one embodiment, the analgesic agent is a topical or systemic analgesic selected from the group consisting of: ibuprofen, diclofenac, capsaicin, lidocaine, and pramoxine HCl.

In one embodiment, the analgesic agent is pramoxine HCl (also referred to as pramocaine HCl, INN, or BAN). In one embodiment, the pharmaceutical composition comprises an amount of pramoxine HCl that is less than the generally recognized therapeutically effective amount. In one embodiment, the pharmaceutical composition comprises an amount of pramoxine HCl that is equal to or greater than the generally recognized therapeutically effective amount. In one embodiment, the pharmaceutical composition comprises about 1 mg to about 20 mg of pramoxine HCl per gram of pharmaceutical composition, for example about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, or about 20 mg of pramoxine HCl per gram of pharmaceutical composition.

In one embodiment, one gram of pharmaceutical composition comprises about 3.5 mg of neomycin sulfate, about 10,000 units of polymyxin B, about 500 units of bacitracin zinc, and about 1 mg to about 20 mg of pramoxine HCl. In one embodiment, one gram of pharmaceutical composition comprises about 3.5 mg of neomycin sulfate, about 10,000 units of polymyxin B, about 500 units of bacitracin zinc, and about 1 mg to less than about 10 mg of pramoxine HCl. In one embodiment, one gram of pharmaceutical composition comprises about 3.5 mg of neomycin sulfate, about 10,000 units of polymyxin B, about 500 units of bacitracin zinc, and about 10 mg of pramoxine HCl. In one embodiment, one gram of pharmaceutical composition comprises about 3.5 mg of neomycin sulfate, about 10,000 units of polymyxin B, no bacitracin zinc, and about 1 mg to about 20 mg of pramoxine HCl. In one embodiment, one gram of pharmaceutical composition comprises about 3.5 mg of neomycin sulfate, about 10,000 units of polymyxin B, no bacitracin zinc, and about 1 mg to less than about 10 mg of pramoxine HCl. In one embodiment, one gram of pharmaceutical composition comprises about 3.5 mg of neomycin sulfate, about 10,000 units of polymyxin B, no bacitracin zinc, and about 10 mg of pramoxine HCl.

Any pharmaceutically acceptable excipient known to those of skill in the art may be used in pharmaceutical compositions according to the present disclosure. Any excipient selected for use in the therapeutic and cosmetic compositions should be pharmaceutically and/or cosmetically acceptable and appropriate for the form in which the therapeutic composition will be used, e.g., cream, gel, milk, oil, lotion, paste and the like. Preferably, the excipient has an affinity for the skin or gingiva, is well tolerated, and stable when used in an amount adequate to provide the desired consistency and ease of application. By way of example only, a pharmaceutical composition according to the present disclosure may comprise one or more of: surfactants, preservatives, flavouring agents, co-solvents, viscosity aids, suspension aids, and lipophilic phases. In one embodiment, the pharmaceutical composition comprises one or more of: cocoa butter, cottonseed oil, olive oil, sodium pyruvate, vitamin E, white petrolatum, emulsifying wax, methylparaben, mineral oil, poloxamer 188, propylene glycol, and purified water. In one embodiment, the pharmaceutical composition comprises cocoa butter, cottonseed oil, olive oil, sodium pyruvate, vitamin E, and white petrolatum. In one embodiment, the pharmaceutical composition comprises white petrolatum. In one embodiment, the pharmaceutical composition comprises emulsifying wax, methylparaben, mineral oil, poloxamer 188, propylene glycol, purified water, and white petrolatum.

In one embodiment, the pharmaceutical composition comprises about 0.5 wt. % to about 5 wt. % of a surfactant such as an ethoxylated natural fatty alcohol (e.g., Steareth-2), for example, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.05 wt. %, about 1.1 wt. %, about 1.15 wt. %, about 1.2 wt. %, about 1.25 wt. %, about 1.3 wt. %, about 1.35 wt. %, about 1.4 wt. %, about 1.45 wt. %, about 1.5 wt. %, about 1.55 wt. %, about 1.6 wt. %, about 1.65 wt. %, about 1.7 wt. %, about 1.75 wt. %, about 1.8 wt. %, about 1.85 wt. %, about 1.9 wt. %, about 1.95 wt. %, about 2 wt. %, about 2.05 wt. %, about 2.1 wt. %, about 2.15 wt. %, about 2.2 wt. %, about 2.25 wt. %, about 2.3 wt. %, about 2.35 wt. %, about 2.4 wt. %, about 2.45 wt. %, about 2.5 wt. %, about 2.55 wt. %, about 2.6 wt. %, about 2.65 wt. %, about 2.7 wt. %, about 2.75 wt. %, about 2.8 wt. %, about 2.85 wt. %, about 2.9 wt. %, about 2.95 wt. %, about 3 wt. %, about 3.05 wt. %, about 3.1 wt. %, about 3.15 wt. %, about 3.2 wt. %, about 3.25 wt. %, about 3.3 wt. %, about 3.35 wt. %, about 3.4 wt. %, about 3.45 wt. %, about 3.5 wt. %, about 3.55 wt. %, about 3.6 wt. %, about 3.65 wt. %, about 3.7 wt. %, about 3.75 wt. %, about 3.8 wt. %, about 3.85 wt. %, about 3.9 wt. %, about 3.95 wt. %, about 4 wt. %, about 4.05 wt. %, about 4.1 wt. %, about 4.15 wt. %, about 4.2 wt. %, about 4.25 wt. %, about 4.3 wt. %, about 4.35 wt. %, about 4.4 wt. %, about 4.45 wt. %, about 4.5 wt. %, about 4.55 wt. %, about 4.6 wt. %, about 4.65 wt. %, about 4.7 wt. %, about 4.75 wt. %, about 4.8 wt. %, about 4.85 wt. %, about 4.9 wt. %, about 4.95 wt. %, about 5 wt. % of the surfactant. In one embodiment the surfactant is Steareth-2 (e.g., BRIJ S2, Croda International plc).

In one embodiment, the pharmaceutical composition comprises about 0.5 wt. % to about 5 wt. % of an emulsifier such as a polyoxyethylene fatty ether (e.g., Steareth-21), for example, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.05 wt. %, about 1.1 wt. %, about 1.15 wt. %, about 1.2 wt. %, about 1.25 wt. %, about 1.3 wt. %, about 1.35 wt. %, about 1.4 wt. %, about 1.45 wt. %, about 1.5 wt. %, about 1.55 wt. %, about 1.6 wt. %, about 1.65 wt. %, about 1.7 wt. %, about 1.75 wt. %, about 1.8 wt. %, about 1.85 wt. %, about 1.9 wt. %, about 1.95 wt. %, about 2 wt. %, about 2.05 wt. %, about 2.1 wt. %, about 2.15 wt. %, about 2.2 wt. %, about 2.25 wt. %, about 2.3 wt. %, about 2.35 wt. %, about 2.4 wt. %, about 2.45 wt. %, about 2.5 wt. %, about 2.55 wt. %, about 2.6 wt. %, about 2.65 wt. %, about 2.7 wt. %, about 2.75 wt. %, about 2.8 wt. %, about 2.85 wt. %, about 2.9 wt. %, about 2.95 wt. %, about 3 wt. %, about 3.05 wt. %, about 3.1 wt. %, about 3.15 wt. %, about 3.2 wt. %, about 3.25 wt. %, about 3.3 wt. %, about 3.35 wt. %, about 3.4 wt. %, about 3.45 wt. %, about 3.5 wt. %, about 3.55 wt. %, about 3.6 wt. %, about 3.65 wt. %, about 3.7 wt. %, about 3.75 wt. %, about 3.8 wt. %, about 3.85 wt. %, about 3.9 wt. %, about 3.95 wt. %, about 4 wt. %, about 4.05 wt. %, about 4.1 wt. %, about 4.15 wt. %, about 4.2 wt. %, about 4.25 wt. %, about 4.3 wt. %, about 4.35 wt. %, about 4.4 wt. %, about 4.45 wt. %, about 4.5 wt. %, about 4.55 wt. %, about 4.6 wt. %, about 4.65 wt. %, about 4.7 wt. %, about 4.75 wt. %, about 4.8 wt. %, about 4.85 wt. %, about 4.9 wt. %, about 4.95 wt. %, about 5 wt. % of the emulsifier. In one embodiment the emulsifier is Steareth-21 (e.g., BRIJ S721, Croda International plc).

In one embodiment, the pharmaceutical composition comprises a stabilizer such as a cetyl alcohol or a saturated cetyl alcohol (e.g., cetyl alcohol). In one embodiment, the pharmaceutical composition comprises about 0.1 wt. % to about 5 wt. % of a stabilizer, for example about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.01 wt. %, about 1.02 wt. %, about 1.03 wt. %, about 1.04 wt. %, about 1.05 wt. %, about 1.06 wt. %, about 1.07 wt. %, about 1.08 wt. %, about 1.09 wt. %, about 1.1 wt. %, about 1.11 wt. %, about 1.12 wt. %, about 1.13 wt. %, about 1.14 wt. %, about 1.15 wt. %, about 1.16 wt. %, about 1.17 wt. %, about 1.18 wt. %, about 1.19 wt. %, about 1.2 wt. %, about 1.21 wt. %, about 1.22 wt. %, about 1.23 wt. %, about 1.24 wt. %, about 1.25 wt. %, about 1.26 wt. %, about 1.27 wt. %, about 1.28 wt. %, about 1.29 wt. %, about 1.3 wt. %, about 1.31 wt. %, about 1.32 wt. %, about 1.33 wt. %, about 1.34 wt. %, about 1.35 wt. %, about 1.36 wt. %, about 1.37 wt. %, about 1.38 wt. %, about 1.39 wt. %, about 1.4 wt. %, about 1.41 wt. %, about 1.42 wt. %, about 1.43 wt. %, about 1.44 wt. %, about 1.45 wt. %, about 1.46 wt. %, about 1.47 wt. %, about 1.48 wt. %, about 1.49 wt. %, about 1.5 wt. %, about 1.51 wt. %, about 1.52 wt. %, about 1.53 wt. %, about 1.54 wt. %, about 1.55 wt. %, about 1.56 wt. %, about 1.57 wt. %, about 1.58 wt. %, about 1.59 wt. %, about 1.6 wt. %, about 1.61 wt. %, about 1.62 wt. %, about 1.63 wt. %, about 1.64 wt. %, about 1.65 wt. %, about 1.66 wt. %, about 1.67 wt. %, about 1.68 wt. %, about 1.69 wt. %, about 1.7 wt. %, about 1.71 wt. %, about 1.72 wt. %, about 1.73 wt. %, about 1.74 wt. %, about 1.75 wt. %, about 1.76 wt. %, about 1.77 wt. %, about 1.78 wt. %, about 1.79 wt. %, about 1.8 wt. %, about 1.81 wt. %, about 1.82 wt. %, about 1.83 wt. %, about 1.84 wt. %, about 1.85 wt. %, about 1.86 wt. %, about 1.87 wt. %, about 1.88 wt. %, about 1.89 wt. %, about 1.9 wt. %, about 1.91 wt. %, about 1.92 wt. %, about 1.93 wt. %, about 1.94 wt. %, about 1.95 wt. %, about 1.96 wt. %, about 1.97 wt. %, about 1.98 wt. %, about 1.99 wt. %, about 2 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt % of the stabilizer. In one embodiment, the stabilizer is cetyl alcohol (e.g., Crodacol C95 EP, Croda International plc).

In one embodiment, the pharmaceutical composition comprises one or more antioxidants such as ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, niacinamide, and the like. In one embodiment, the pharmaceutical composition comprises about 0.01 wt. % to about 2 wt. % of an antioxidant, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. % of the one or more antioxidant.

In one embodiment the antioxidant is ascorbyl palmitate. In one embodiment the antioxidant is α-tocopherol. In one embodiment the antioxidant is ascorbic acid. In one embodiment the antioxidant is idebenone. In one embodiment, the antioxidant is ubiquinone. In one embodiment, the antioxidant is ferulic acid. In one embodiment, the antioxidant is coenzyme Q10. In one embodiment, the antioxidant is lycopene. In one embodiment, the antioxidant is green tea. In one embodiment, the antioxidant is catechins. In one embodiment, the antioxidant is epigallocatechin 3-gallate (EGCG). In one embodiment, the antioxidant is green tea polyphenols (GTP). In one embodiment, the antioxidant is silymarin. In one embodiment, the antioxidant is coffeeberry. In one embodiment, the antioxidant is resveratrol. In one embodiment, the antioxidant is grape seed. In one embodiment, the antioxidant is pomegranate extracts. In one embodiment, the antioxidant is genisten. In one embodiment, the antioxidant is pycnogenol. In one embodiment, the antioxidant is niacinamide. In one embodiment, the pharmaceutical composition comprises about 0.01 wt. % to about 0.5 wt. % of one or more antioxidants selected from the group consisting of ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, and niacinamide. In one embodiment, the pharmaceutical composition comprises about 0.1 wt. % to about 0.3 wt. % of one or more antioxidants selected from the group consisting of ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, and niacinamide. In one embodiment, the pharmaceutical composition comprises about 0.3 wt. % to about 0.5 wt. % of one or more antioxidants selected from the group consisting of ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, and niacinamide. In one embodiment, the pharmaceutical composition comprises about 0.45 wt. % of one or more antioxidants selected from the group consisting of ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, COFFEEBERRY, resveratrol, grape seed, pomegranate extracts, genisten, pycnogenol, and niacinamide. In one embodiment, the pharmaceutical composition comprises about 0.05 wt. % of idebenone. In one embodiment, the pharmaceutical composition comprises about 0.05 wt. % to about 1 wt. % of ubiquinone, for example about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, or about 1 wt. % of ubiquinone. In one embodiment, the pharmaceutical composition comprises about 0.1 wt. % to about 1 wt. % of ferulic acid, for example about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, or about 1 wt. % of ferulic acid. In one embodiment, the pharmaceutical composition comprises about 0.01 wt. % to about 0.5 wt. % of ascorbyl palmitate, about 0.01 wt. % to about 0.5 wt. % of α-tocopherol, and about 0.01 wt. % to about 0.5 wt. % of ascorbic acid. In one embodiment the pharmaceutical composition comprises about 0.1 wt. % to about 0.3 wt. % of ascorbyl palmitate, about 0.1 wt. % to about 0.3 wt. % of α-tocopherol, and about 0.05 wt. % to about 0.2 wt. % of ascorbic acid. In one embodiment the pharmaceutical composition comprises about 0.2 wt. % of ascorbyl palmitate, about 0.15 wt. % of α-tocopherol, and about 0.1 wt. % of ascorbic acid.

In one embodiment, the pharmaceutical composition comprises one or more emollients such as a fully saturated triglyceride (e.g., medium-chain triglycerides such as Crodamol GTCC, Croda International plc), myristyl myristate, isopropryl palmitate, and glycerin. In one embodiment, the pharmaceutical composition comprises about 0.5 wt. % to about 20 wt. % of an emollient, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, about 10 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13 wt. %, about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, about 14 wt. %, about 14.1 wt. %, about 14.2 wt. %, about 14.3 wt. %, about 14.4 wt. %, about 14.5 wt. %, about 14.6 wt. %, about 14.7 wt. %, about 14.8 wt. %, about 14.9 wt. %, about 15 wt. %, about 15.1 wt. %, about 15.2 wt. %, about 15.3 wt. %, about 15.4 wt. %, about 15.5 wt. %, about 15.6 wt. %, about 15.7 wt. %, about 15.8 wt. %, about 15.9 wt. %, about 16 wt. %, about 16.1 wt. %, about 16.2 wt. %, about 16.3 wt. %, about 16.4 wt. %, about 16.5 wt. %, about 16.6 wt. %, about 16.7 wt. %, about 16.8 wt. %, about 16.9 wt. %, about 17 wt. %, about 17.1 wt. %, about 17.2 wt. %, about 17.3 wt. %, about 17.4 wt. %, about 17.5 wt. %, about 17.6 wt. %, about 17.7 wt. %, about 17.8 wt. %, about 17.9 wt. %, about 18 wt. %, about 18.1 wt. %, about 18.2 wt. %, about 18.3 wt. %, about 18.4 wt. %, about 18.5 wt. %, about 18.6 wt. %, about 18.7 wt. %, about 18.8 wt. %, about 18.9 wt. %, about 19 wt. %, about 19.1 wt. %, about 19.2 wt. %, about 19.3 wt. %, about 19.4 wt. %, about 19.5 wt. %, about 19.6 wt. %, about 19.7 wt. %, about 19.8 wt. %, about 19.9 wt. %, or about 20 wt. % of an emollient. In one embodiment, the pharmaceutical composition comprises about 0.5 wt. % to about 5 wt. % of any one emollient. In one embodiment, the one or more emollients are selected from the group consisting of medium-chain triglycerides (e.g., Crodamol GTCC, Croda International plc), myristyl myristate, isopropryl palmitate, and glycerin.

In one embodiment, the pharmaceutical composition comprises medium-chain triglycerides (e.g., Crodamol GTCC), myristyl myristate, isopropryl palmitate and glycerin in a combined amount of about 0.5 wt. to about 20 wt. %. In one embodiment, the pharmaceutical composition comprises about 0.5 wt. % to about 5 wt. % of medium-chain triglycerides (e.g., Crodamol GTCC), for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of medium-chain triglycerides (e.g., Crodamol GTCC). In one embodiment, the pharmaceutical composition comprises about 0.5 wt. % to about 5 wt. % of myristyl myristate, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of myristyl myristate.

In one embodiment, the pharmaceutical composition comprises about 0.5 wt. % to about 8 wt. % of isopropryl palmitate, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, or about 8 wt. % of isopropryl palmitate.

In one embodiment, the pharmaceutical composition comprises about 0.5 wt. % to about 5 wt. % of glycerin, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of glycerin. in one embodiment, the pharmaceutical composition comprises about 2 wt. % of medium-chain triglycerides (e.g., Crodamol GTCC), about 2 wt. % of myristyl myristate (e.g., Crodamol MM, Croda International plc), about 4 wt. % of isopropryl palmitate (e.g., Crodamol IPP, Croda International plc), and about 1 wt. % of glycerin.

In one embodiment, the pharmaceutical composition comprises a preservative such as phenoxyethanol. In one embodiment, the pharmaceutical composition comprises about 0.1 wt. % to about 5 wt. % of a preservative, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of a preservative. In one embodiment, the preservative is phenoxyethanol. In one embodiment, the pharmaceutical composition comprises about 0.5 wt. % to about 5 wt. % of phenoxyethanol. In one embodiment, the pharmaceutical composition comprises about 0.5 wt. % to about 2 wt. % of phenoxyethanol. In one embodiment, the pharmaceutical composition comprises about 1 wt. % of phenoxyethanol.

In one embodiment, the pharmaceutical composition comprises one or more thickeners, such as a cross-linked polymer (e.g., a cross-linked acrylic acid polymer such as carbomer, available commercially as Carbopol ETD2020NF, Lubrizol Corp.), a polysaccharide (e.g., a xanthan gum such as CPKelko's Keltrol 11K). In one embodiment, the pharmaceutical composition comprises about 0.1 wt. % to about 5 wt. % of one or more thickeners, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of one or more thickeners. In one embodiment, the one or more thickeners is one or more of a cross-linked acrylic acid polymer and a polysaccharide. In one embodiment, the one or more thickeners are Carbopol ETD2020NF and Keltrol 11K. In one embodiment, the pharmaceutical composition comprises about 0.1 wt. % to about 5 wt. % of Carbopol ETD2020NF and about 0.1 wt. % to about 5 wt. % of Keltrol 11K. In one embodiment, the pharmaceutical composition comprises about 0.5 wt. % to about 1 wt. % of Carbopol ETD2020NF and about 0.2 wt. % to about 1 wt. % of Keltrol 11K. In one embodiment, the pharmaceutical composition comprises about 0.8 wt. % of Carbopol ETD2020NF and about 0.4 wt. % of Keltrol 11K.

In one embodiment, the pharmaceutical composition comprises one or more texturizers such as a lecithin (e.g., a liquid soy lecithin such as Leciprime 1400 IPM, Cargill, Inc.). In one embodiment, the pharmaceutical composition comprises about 0.1 wt. % to about 5 wt. % of one or more texturizers, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of one or more texturizers. In one embodiment, the one or more texturizers comprise Leciprime 1400 IPM. In one embodiment, the pharmaceutical composition comprises about 0.1 wt. % to about 5 wt. % of Leciprime 1400 IPM. In one embodiment, the pharmaceutical composition comprises about 0.2 wt. % to about 1 wt. % of Leciprime 1400 IPM. In one embodiment, the pharmaceutical composition comprises about 0.5 wt. % of Leciprime 1400 IPM.

In one embodiment, the pharmaceutical composition comprises one or more fragrances. In one embodiment, the pharmaceutical composition comprises about 0.01 wt. % to about 0.5 wt. % of one or more fragrances, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, or about 0.5 wt. % of one or more fragrances.

In one embodiment, the pharmaceutical composition comprises: about 0.5 wt. % to about 20 wt. % of one or more of DHA, EPA, and GLA; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 0.5 wt. % to about 5 wt. % of one or more surfactants; about 0.5 wt. % to about 5 wt. % of one or more emulsifiers; about 0.05 wt. % to about 5 wt. % of one or more stabilizers; about 0.01 wt. % to about 2 wt. % of one or more antioxidants; about 0.5 wt. % to about 20 wt. % of one or more emollients; about 0.1 wt. % to about 5 wt. % of one or more preservatives; about 0.1 wt. % to about 5 wt. % of one or more thickeners; about 0.1 wt. % to about 5 wt. % of one or more texturizers; and about 0.01 wt. % to about 0.5 wt. % of one or more fragrances.

In one embodiment, the pharmaceutical composition comprises: about 0.1 wt. % to about 20 wt. % of one or more of DHA, EPA, and GLA; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1 wt. % to about 2 wt. % of one or more surfactants; about 1 wt. % to about 2 wt. % of one or more emulsifiers; about 0.1 wt. % to about 1 wt. % of one or more stabilizers; about 0.1 wt. % to about 1 wt. % of one or more antioxidants; about 5 wt. % to about 15 wt. % of one or more emollients; about 0.5 wt. % to about 2 wt. % of one or more preservatives; about 0.5 wt. % to about 2 wt. % of one or more thickeners; about 0.1 wt. % to about 2 wt. % of one or more texturizers; and about 0.01 wt. % to about 0.1 wt. % of one or more fragrances.

In one embodiment, the pharmaceutical composition comprises: about 0.1 wt. % to about 20 wt. % of one or more of DHA, EPA, and GLA; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1.65 wt. % of one or more surfactants; about 1.35 wt. % of one or more emulsifiers; about 0.5 wt. % of one or more stabilizers; about 0.45 wt. % of one or more antioxidants; about 9 wt. % of one or more emollients; about 1 wt. % of one or more preservatives; about 1.2 wt. % of one or more thickeners; about 0.5 wt. % of one or more texturizers; and about 0.05 wt. % of one or more fragrances.

In one embodiment, the pharmaceutical composition comprises: about 0.1 wt. % to about 20 wt. % of one or more of DHA, EPA, and GLA; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 0.5 wt. % to about 5 wt. % of Steareth-2; about 0.5 wt. % to about 5 wt. % of Steareth-21; about 0.1 wt. % to about 5 wt. % of cetyl alcohol; about 0.01 wt. % to about 2 wt. % of a combination of medium-chain triglycerides, myristyl myristate, isopropryl palmitate, and/or glycerin; about 0.5 wt. % to about 20 wt. % of one or more emollients; about 0.1 wt. % to about 5 wt. % of phenoxyethanol; about 0.1 wt. % to about 5 wt. % of a combination of carbomer and/or xanthan gum; about 0.1 wt. % to about 5 wt. % of liquid soy lecithin; and about 0.01 wt. % to about 0.5 wt. % of one or more fragrances.

In one embodiment, the pharmaceutical composition comprises: about 0.1 wt. % to about 20 wt. % of one or more of DHA, EPA, and GLA; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1 wt. % to about 2 wt. % of Steareth-2; about 1 wt. % to about 2 wt. % of Steareth-21; about 0.1 wt. % to about 1 wt. % of cetyl alcohol; about 0.1 wt. % to about 1 wt. % of a combination of ascorbyl palmitate, α-tocopherol, and ascorbic acid; about 5 wt. % to about 15 wt. % of a combination of medium-chain triglycerides, myristyl myristate, isopropryl palmitate, and/or glycerin; about 0.5 wt. % to about 2 wt. % of phenoxyethanol; about 0.5 wt. % to about 2 wt. % of a combination of carbomer and/or xanthan gum; about 0.1 wt. % to about 2 wt. % of liquid soy lecithin; and about 0.01 wt. % to about 0.1 wt. % of one or more fragrances.

In one embodiment, the pharmaceutical composition comprises: about 0.1 wt. % to about 20 wt. % of one or more of DHA, EPA, and GLA; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1.65 wt. % of Steareth-2; about 1.35 wt. % of Steareth-21; about 0.5 wt. % of cetyl alcohol; about 0.2 wt. % of ascorbyl palmitate; about 0.15 wt. % of α-tocopherol; about 0.1 wt. % of ascorbic acid; about 2 wt. % of medium-chain triglycerides; about 2 wt. % of myristyl myristate; about 4 wt. % of isopropryl palmitate; about 1 wt. % of glycerin; about 1 wt. % of phenoxyethanol, about 0.8 wt. % of carbomer; about 0.4 wt. % of xanthan gum; about 0.5 wt. % of liquid soy lecithin; and about 0.05 wt. % of one or more fragrances.

In one embodiment, the pharmaceutical composition comprises: about 0.1 wt. % to about 20 wt. % of one or more of DHA, EPA, and GLA; optionally about 0.01% to about 1.0% mg of triclosan in a pharmaceutical composition;

A composition for use in accordance with the disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In one embodiment, a composition including, for example, a pharmaceutical composition, as disclosed herein is formulated as an aerosol, a gel, an ointment, a lotion, a cream, a gel stick, a liniment, a paste or a spray.

Such formulations may be stable and comprise an amount (e.g., a therapeutically effective amount) of DHA, EPA, GLA in combination with one or more antibacterial agents selected from the group consisting of: nicotinamide, triclosan, metronidazole, neomycin sulfate, polymyxin B and bacitracin zinc.

The present disclosure also provides the disclosed compositions or formulations as a component in a product for use in the treatment of skin or ocular infections. In one embodiment, the product comprises a container and a pharmaceutical composition comprising a therapeutically effective amount of DHA, EPA, GLA, or a combination thereof. In one embodiment, the pharmaceutical composition comprises from about 0.1 wt. % to about 20 wt. % of DHA, EPA, GLA, or a combination thereof. In one embodiment, the product comprises a pharmaceutical composition as disclosed herein.

Methods of Treatment of Diseases and/or Disorders

The compositions and formulations disclosed herein may be used in the prevention and treatment of diseases and/or disorders including, for example, disease and/or disorders of the skin and gingiva such as contusions, wounds, burns, sores, ulcers, scrapes, incisions, lacerations, conjunctivitis, stye, blepharitis, skin infection, gingivitis or periodontal disease.

Methods are provided herein for treating or preventing skin or ocular infections in a subject in need thereof comprising administering to the subject a pharmaceutical composition comprising an effective amount including, for example, a therapeutically effective amount (e.g., 0.1 wt. % to about 20 wt. %) of DHA, EPA, or GLA as described herein.

The term “skin infection” herein refers to any disease or disorder of the skin that presents with one or more occurring or reoccurring symptoms such as erythema, warmth, swelling, tenderness, pain, ulcers, lesion(s), nodules, fever, scaling, plaques, papules, pustules, cysts, and the like. Non-limiting examples of skin infections include cellulitis; erysipelas; impetigo; folliculitis; furuncles; carbuncles; secondarily infected dermatoses such as atopic dermatitis, allergic contact dermatitis and psoriasis; secondarily infected traumatic lesions; acne; and other skin disorders associated with infectious pathogens.

In one embodiment, the present disclosure provides a method of treating or preventing a skin or ocular infection in a subject in need thereof. In one embodiment, the method comprises administering to the subject a pharmaceutical composition as disclosed herein, for example a pharmaceutical comprising a therapeutically effective amount of DHA, EPA, GLA, or a combination thereof, along with one or more antibiotic agents. In one embodiment, the pharmaceutical composition comprises from about 0.1 wt. % to about 20 wt. % of DHA, EPA, GLA, or a combination thereof.

In one embodiment, the present disclosure provides a method of inhibiting one or more skin or ocular pathogens including, for example, its growth, colonization and/or infection in a subject in need thereof. In one embodiment, the method comprises contacting a skin or ocular pathogen with a composition as disclosed herein, for example a composition comprising one or more of DHA, EPA, and GLA and one or more antibiotic agents. In one embodiment, the skin or ocular pathogen is one or more of: Staphylococcus spp. (including, for example, S. aureus, S. lugdunensis, S. schleiferi and other coagulase-negative Staphylococcus spp.), Streptococcus spp. (including, for example, β-haemolytic Streptococci, Viridans group Streptococci, non-haemolytic Streptococci, and Streptococcus milleri group), Corynebacterium spp., Bacillus spp. (including, for example, B. anthracis and B. cereus), Acinetobacter spp., Moraxella spp., Peptostreptococcus spp., Propionibacterium spp., (including, for example, P. Acnes), Candida spp., Pseudomonas spp. and other non-fermentative bacilli (including, for example, P. aeruginosa), Dermatophytes, Enterobacteriaceae, Pasturella multocida, Mycobacterium spp., Haemophilus spp., Nocardia spp., Erysipelothrix rhusiopathiae, Vibrio spp., Enterococcus spp., Eikenella corrodens, anaerobes, Corynebacterium spp., Actinomyces spp., and/or fungal pathogens. In one embodiment, the composition comprises from about 0.1 wt. % to about 20 wt. % of DHA, EPA, GLA, or a combination thereof.

In one embodiment, the method comprises topically administering the pharmaceutical composition to an area of the skin or eye area infected by a pathogen and/or to an area of the skin or eye that is generally prone to development of an infection and/or previously had an infection.

In one embodiment, the method comprises administering a pharmaceutical composition as disclosed herein once per day, twice per day, three times per day, or more than three times per day.

In one embodiment, upon treatment in accordance with the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the treated area of the skin or gingiva comprises about 10%, about 20%, about 30%, about 40%, about 50%, about 60%, about 70%, about 80%, about 90%, or greater than about 90% fewer lesions than before treatment.

As used herein, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein, such as DHA, EPA, and/or GLA, is an amount effective to achieve a desired pharmacologic effect or therapeutic improvement without undue adverse side effects. In other embodiments, it is understood that “an effect amount” or “a therapeutically effective amount” varies from subject to subject, due to variation in metabolism, age, weight, general condition of the subject, the condition being treated, the severity of the condition being treated, and the judgment of the prescribing physician. The term “pharmaceutically acceptable” in the present context means that the substance in question does not produce unacceptable toxicity to the subject or interaction with other components of the composition.

In another embodiment, the present disclosure provides a method of slowing progression of or promoting regression of a skin or ocular infection in a subject in need thereof, comprising administering to a subject in need thereof one or more compositions as disclosed herein.

In one embodiment, the present disclosure provides a method of reducing or preventing side effects associated with topical administration of neomycin sulfate. Administration of high doses of neomycin sulfate has been associated with redness and irritation of the skin, allergic reactions (e.g., rash, hives, itching, difficult breathing, chest tightness, swelling of the mouth, face, lips or tongue), bloody stool, dizziness, hearing loss, muscle twitching, ringing in the ears, seizures, tingling or numbness of the skin, vaginal irritation or discharge, and stomach pains or cramps. In one embodiment, a method of reducing side effects associated with topical administration of neomycin sulfate comprises discontinuing administration of a first pharmaceutical composition comprising neomycin sulfate and administering to a subject a second pharmaceutical composition as disclosed herein. In one embodiment, the second pharmaceutical composition includes an amount of neomycin sulfate that is less than the amount of neomycin sulfate in the first pharmaceutical composition. In one embodiment, the second pharmaceutical composition includes an amount of neomycin sulfate that is about equal to or equal to the amount of neomycin sulfate in the first pharmaceutical composition. In one embodiment, the second pharmaceutical composition includes an amount of neomycin sulfate that is more than the amount of neomycin sulfate in the first pharmaceutical composition. In one embodiment, the second pharmaceutical composition includes no neomycin sulfate, essentially no neomycin sulfate, or substantially no neomycin sulfate.

In one embodiment, the present disclosure provides a method of reducing or preventing side effects associated with topical administration of polymyxin B. Administration of high doses of polymyxin B has been associated with redness and irritation of the skin, severe allergic reactions (e.g., rash, hives, itching, difficulty breathing, chest tightness, swelling of the mouth, face, lips, or tongue), changes in the amount of urine, changes in hearing or ringing in the ears, dizziness, drowsiness, flushing of the face, loss of coordination, mental or mood changes (e.g., irritability), severe headaches, stiff neck, tingling or numbness in the mouth, hands, or feet, unusual weakness, unusually fast heartbeat, and changes in vision. In one embodiment, a method of reducing side effects associated with topical administration of polymyxin B comprises discontinuing administration of a first pharmaceutical composition comprising polymyxin B and administering to a subject a second pharmaceutical composition as disclosed herein. In one embodiment, the second pharmaceutical composition includes an amount of polymyxin B that is less than the amount of polymyxin B in the first pharmaceutical composition. In one embodiment, the second pharmaceutical composition includes an amount of polymyxin B that is about equal to or equal to the amount of polymyxin B in the first pharmaceutical composition. In one embodiment, the second pharmaceutical composition includes an amount of polymyxin B that is more than the amount of polymyxin B in the first pharmaceutical composition. In one embodiment, the second pharmaceutical composition includes no polymyxin B, essentially no polymyxin B, or substantially no polymyxin B.

In one embodiment, the present disclosure provides a method of reducing or preventing side effects associated with topical administration of bacitracin zinc. Administration of high doses of bacitracin zinc has been associated with redness and irritation of the skin, severe allergic reactions (e.g., rash, hives, itching, difficulty breathing, chest tightness, swelling of the mouth, face, lips, or tongue), changes in vision, continued redness, burning, or itching, eye pain, and secondary infection. In one embodiment, a method of reducing side effects associated with topical administration of bacitracin zinc comprises discontinuing administration of a first pharmaceutical composition comprising bacitracin zinc and administering to a subject a second pharmaceutical composition as disclosed herein. In one embodiment, the second pharmaceutical composition includes an amount of bacitracin zinc that is less than the amount of bacitracin zinc in the first pharmaceutical composition. In one embodiment, the second pharmaceutical composition includes an amount of bacitracin zinc that is about equal to or equal to the amount of bacitracin zinc in the first pharmaceutical composition. In one embodiment, the second pharmaceutical composition includes an amount of bacitracin zinc that is more than the amount of bacitracin zinc in the first pharmaceutical composition. In one embodiment, the second pharmaceutical composition includes no bacitracin zinc, essentially no bacitracin zinc, or substantially no bacitracin zinc.

In one embodiment, the present disclosure provides a method of reducing scarring in at least a portion of a subject's skin. In one embodiment, the method comprises administering to the subject a therapeutically effective amount of a pharmaceutical composition as disclosed herein. In one embodiment, the amount of scarring per square inch for a given affected area of the subject's skin after administration of a pharmaceutical composition as disclosed herein is less than, or substantially less than the amount of scarring present in the same area of skin before administration of a pharmaceutical composition as disclosed herein. In one embodiment, treatment according to the present method results in a 10% reduction, about a 20% reduction, about a 30% reduction, about a 40% reduction, about a 50% reduction, about a 60% reduction, about a 70% reduction, about a 80% reduction, about a 90% reduction, or more than a 90% reduction in scarring for a given area of the subject's skin. In one embodiment, the reduction in scarring occurs within about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months of the initiation of the treatment method.

The present disclosure also provides methods of improving the antimicrobial activity of an agent used in the treatment of acne. The term “antimicrobial agent” includes antibiotics and antifungals. More specifically, “antimicrobial agents” may include neomycin sulfate, polymyxin B, bacitracin zinc, β-lactams (e.g., ampicillin, amoxicillin, imipenem, meropenem), carbapenems, cephalosporins (e.g., cephalexin, cephalothin, cefalozin, cefuroxime, cefotaxime, ceftazidime), fluoroquinolones, oxazolidinones, lincosamides, metronidazole, macrolide antibiotics (e.g., clindamycin, erythromycin), quinolone anibiotics (e.g., levofloxacin, ciprofloxacin), penicillins, glycopeptides (e.g., vancomycin), aminoglycosides (e.g., neomycin, gentamicin, tobramycin), trimethoprim/sulfamethoxazole (also known as co-trimoxazole or TMP/SMX), triclosan, doxycycline and tetracycline. In one embodiment, the method comprises adding a pharmaceutical comprising one or more of DHA, EPA, and GLA to the agent. In one embodiment, the agent is one in which no previous antimicrobial activity was appreciated. In one embodiment, the pharmaceutical composition is a pharmaceutical composition as disclosed herein, for example a pharmaceutical composition comprising from about 0.1 wt. % to about 20 wt. % of DHA, EPA, GLA, or a combination thereof.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### Example 1

### Effect of Various Compounds on the Growth of P. Acnes and S. aureus

### Reagents and Bacteria

All bacterial culture media and reagents were of the highest purity available and purchased from Sigma-Aldrich Ltd unless stated. Stock solutions of BPO (75%) were prepared in DMSO (≧99.9%) to 50 mg/mL; POL and FUS were prepared in sterile distilled water to 20 mg/mL; while DHA, EPA, GLA, MUP and SA were prepared in ethanol to 50 mg/mL. NEO was purchased as a 10 mg/mL solution in sterile saline. P. acnes NCTC737 was purchased from the National Collection of Type Cultures (Porton Down, UK). Ten strains of S. aureus were used: two community-acquired methicillin-resistant (MRSA) clinical isolates (CA3 and NRS384), one hospital-acquired MRSA clinical isolate (HA204), two vancomycin-intermediate resistant clinical isolates (5827 and 5836), the reference MRSA strain ATCC43300 (gifted by Dr Peter Warn, University of Manchester, UK), the ‘laboratory’ MRSA strain BB270 and three ‘laboratory’ meticillin-susceptible strains (Newman, SH1000 and RN4220). S. aureus strains were sourced as described in Desbois et al. [52].

### MIC and MBC Determinations

MICs and MBCs were determined for the three LC-PUFA and various clinical antimicrobial agents against P. acnes and S. aureus by broth micro-dilution according to protocols M7-A5 and M11-A5 published by the Clinical and Laboratory Standards Institute [53]. Doubling dilutions of the test compounds were prepared in sterile 96-well plates up to 4096 mg/L. S. aureus experiments were performed in Mueller-Hinton broth (MHB), while P. acnes studies used supplemented brucella broth (SBB). Inoculums of S. aureus were prepared from late logarithmic phase cultures (˜18 h) that had been grown in 5 mL MHB at 37° C. with agitation at 140 rpm, while the P. acnes inoculum was prepared in 2-3 mL SBB by re-suspending colonies from supplemented brucella agar (SBA) plates that had been grown anaerobically (10% CO₂, 10% H₂, 80% N₂) for ˜72 h at 37° C. (A85 Workstation; Don Whitley Scientific Ltd., Shipley, UK). Absorbance readings at 540 nm and 570 nm of the P. acnes and S. aureus suspensions were determined respectively, and bacterial inoculums were adjusted to 1×10⁷ CFU/mL with fresh medium. Inoculum sizes were confirmed by diluting in phosphate-buffered saline (PBS) and plating on Mueller-Hinton agar (MHA) or SBA. Each well was inoculated with 5 μL of bacterial suspension. Control wells contained media inoculated with bacterial suspension only while negative control wells contained uninoculated culture medium only. Further inoculated wells controlled for the antimicrobial effects of the greatest concentrations of DMSO and ethanol used in the test wells. Microtitre plates were incubated at 37° C. for 18-20 h (48 h for P. acnes), and then the MICs were determined to be the lowest concentrations of each compound that prevented visible bacterial growth. MBCs were determined by plating 50 μL from each well showing no visible growth on to MHA or SBA and incubating these plates at 37° C. for 48 h (96 h for P. acnes). The MBCs were the lowest concentrations of each compound that killed ≧99.9% of the initial inoculum. MIC and MBC determinations were performed in duplicate. MIC₅₀ and MBC₅₀ were obtained by performing MICs and MBCs against the ten S. aureus isolates described above, and then determining the concentration required to inhibit or kill 50% of the strains.

### Kill Kinetics

A S. aureus ATCC43300 inoculum was prepared as above, except that PBS was used to adjust the suspension to 1×10⁷ CFU/mL. The inoculum was diluted and plated on MHA to provide an accurate indication of CFU/mL. Solutions in PBS of the three LC-PUFAs were prepared at 1×MBC and 300 μL of each solution was dispensed to separate Eppendorf tubes in triplicate. Controls contained just DMSO and ethanol diluted in PBS to the greatest concentration of each solvent used. Each tube was inoculated with bacterial suspension (15 μL) and incubated statically at 37° C. At 15 min, 30 min, 1 h and 2 h, 10 μL from each tube was plated on MHA in duplicate. Plates were incubated at 37° C. for 24 h and then CFU were quantified.

### Checkerboard Tests

Checkerboard assays were performed according to the standard protocol published by the American Society for Microbiology [54] to investigate the existence of synergistic antimicrobial interactions between the three LC-PUFAs and two anti-acne agents (BPO and SA) against P. acnes. Further checkerboards were performed against S. aureus for BPO, SA and four antibiotics used to treat topical staphylococcal infections (FUS, MUP, NEO and POL) with these agents being assessed separately against each of the three LC-PUFAs. Doubling dilutions of each compound were prepared in TSA or SBB and dispensed to 96-well plates in the standard checkerboard pattern. Concentrations of each compound ranged from ≦ 1/16×MIC to ≧4×MIC. Control wells were dispensed with medium supplemented with the greatest concentration of solvent used in any well, while negative control wells contained just medium. Inoculums were prepared to 1×10⁷ CFU/mL as above. Each well (except for negative controls) was inoculated with 5 μL of bacterial suspension. Plates were incubated as for MIC determinations above and then the presence of bacterial growth was determined for each well according to the clarity of the medium and/or the presence of suspended colonies. The FIC for each well was determined for each interaction and the lowest FICs from duplicate checkerboard assays were used to calculate a mean value that permitted interpretation of the nature of interaction between each combination of compounds

TABLE 1
MICs and MBCs against P. acnes NCTC737
and S. aureus ATCC43300 for three LC-PUFAs.

| | P. acnes | | S. aureus | |
|---|---|---|---|---|
| Compound | MIC (mg/L) | MBC (mg/L) | MIC (mg/L) | MBC (mg/L) |
| DHA | 32 | >4096 | 128 | 128 |
| EPA | 128 | >4096 | 128 | 256 |
| GLA | 64 | >4096 | 512 | 512 |

TABLE 2
MIC₅₀ and MBC₅₀ values for three LC-PUFAs
against ten diverse isolates of S. aureus.

| | S. aureus | |
|---|---|---|
| Compound | MIC₅₀ (mg/L) | MBC₅₀ (mg/L) |
| DHA | 128 | 256 |
| EPA | 256 | 256 |
| GLA | 512 | 512 |

TABLE 3
MICs and MBCs against P. acnes and S. aureus for various clinical
antimicrobial agents used to treat acne or superficial topical staphylococcal
infections. n/d, not determined as these agents are not used against P. acnes.

| | P. acnes | | S. aureus | |
|---|---|---|---|---|
| Compound | MIC (mg/L) | MBC (mg/L) | MIC (mg/L) | MBC (mg/L) |
| BPO | 64 | >4096 | 512 | 512 |
| FUS | n/d | n/d | 0.25 | 8 |
| MUP | n/d | n/d | 0.25 | 16 |
| NEO | n/d | n/d | 32 | 128 |
| POL | n/d | n/d | 16 | 128 |
| SA | 64 | >4096 | 1024 | 2048 |

Using the pre-determined MIC values, checkerboard assays were performed to detect the interactions between the LC-PUFAs and the clinical antimicrobial drugs against the growth of S. aureus. The lowest fractional inhibitory concentration (FIC) on duplicate checkerboards was used to calculate a mean FIC for each interaction, and a mean FIC ≦0.5 was defined as synergy while a mean FIC ≧4 was defined as antagonism. Against S. aureus, none of the combinations gave a mean FIC ≧4, indicating no antagonism of efficacy between the LC-PUFAs and the clinical antimicrobial agents (Table 4). Interestingly combinations of NEO and the LC-PUFAs showed antimicrobial synergy against S. Aureus. (Table 4)

TABLE 4
ΣFIC values of interactions between three LC-PUFAs
and six topical antimicrobial agents against S. aureus
showing that there was synergy (FIC ≦0.5) between
BPO and three LC-PUFAs and NEO and five LC-PUFAs.
Values in bold indicate synergy; n = 2; mean ± one standard deviation.

| S. aureus | EPA | DHA | GLA |
|---|---|---|---|
| BPO | 1.00 ± 0.00 | 2.00 ± 0.00 | 0.56 ± 0.09 |
| SA | 0.88 ± 0.18 | 1.25 ± 0.35 | 0.88 ± 0.18 |
| FUS | 1.13 ± 0.18 | 2.00 ± 0.00 | 1.50 ± 0.71 |
| MUP | 2.00 ± 0.00 | 1.50 ± 0.00 | 2.25 ± 0.35 |
| NEO | 0.38 ± 0.00 | 0.56 ± 0.09 | 0.19 ± 0.00 |
| POL | 2.00 ± 0.00 | 2.00 ± 0.00 | 1.06 ± 0.62 |

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

What is claimed is:

1. A pharmaceutical composition comprising:
DHA, EPA, or GLA; and
a therapeutically effective amount of one or more antibiotic agents.

2. The pharmaceutical composition of claim 1 wherein the antibiotic agent is neomycin.

3. A pharmaceutical composition comprising:
GLA; and
a therapeutically effective amount of one or more antibiotic agents.

4. The pharmaceutical composition of claim 3 wherein the antibiotic is neomycin.

5. A pharmaceutical composition comprising:
GLA; and
a subtherapeutic dose of neomycin
